(19) United States
(12) Patent Application Publication
(10) Pub. No.: US 2013/0004367 A1
(43) Pub. Date: Jan. 3, 2013

(54) SANITIZATION METHOD AND APPARATUS

(75) Inventor: Jon L. ROBERTS, Great Falls, VA (US)

(73) Assignee: VIRWALL SYSTEMS, INC., Lakeville, CT (US)

(21) Appl. No.: 13/538,961

(22) Filed: Jun. 29, 2012

Related U.S. Application Data
(60) Provisional application No. 61/503,964, filed on Jul. 1, 2011.

(51) Int. Cl. A61L 2/10

(52) U.S. Cl. 422/24; 250/494.1; 250/504 R

(57) ABSTRACT

A sanitization apparatus having ultraviolet sanitization in an enclosed container to kill bacteria and other disease carrying organisms. A horizontal or vertical sanitization unit dimensioned to fit objects of varying sizes is provided. An ultraviolet source within the sanitization unit irradiates an object with UV light which, in one embodiment generates ozone gas, thereby killing any microorganisms that might reside on the object that is being sanitized. The ultraviolet light may be installed in either the base or the cover of the sanitization unit. The base and the cover may include reflective surfaces to allow ultraviolet light to reflect and reach different angles and sides of an object that is being sanitized.

### CROSS REFERENCE TO RELATED APPLICATIONS

This application claims the benefit of priority to U.S. Provisional Patent Application Ser. No. 61/503,964 entitled “Sanitization Method and Apparatus” and filed Jul. 1, 2011, which is incorporated herein by reference in its entirety.

### FIELD OF THE INVENTION

The present invention relates generally to sanitization devices and methods. More particularly this invention relates to a device for sterilizing electronic devices and their accessories using ultraviolet radiation and potentially ozone.

### BACKGROUND

It has long been known that germs are spread by, among other things, hand to hand contact. Hence, there's been much in the literature recently concerning the washing of hands in order to prevent the spreading of the common cold as well as other microbes. Further, it has long been known that toothbrushes can be a source of the spreading of germs as well. Recently, it has been discovered that bacteria continue to live on objects used by individuals. Similarly disease causing germs can live on many surfaces and therefore can be a vector for the spread of disease.

### SUMMARY

The various embodiment sanitization units may include a sanitization container comprising a sanitizer base and a sanitizer cover. In the various embodiments, the sanitization units may include “passive,” sanitizer bases that reflect ultraviolet (UV) but do not themselves generate UV energy or “active” sanitizer bases that comprise their own UV sources and do not solely rely on reflection of UV energy from an active UV cover.

According to an embodiment, a sanitization unit with a passive sanitizer base may include a base with a reflective surface and a support that is capable of supporting physical objects on the base with the base unit hingedly, removably or fixedly attached to the base in a manner to create a space between the reflective surface and the support. An ultraviolet (UV) light source may be attached to the interior surface of the cover which also has a reflective surface near the UV light source. UV radiation from the UV light source located in the cover is directed downward toward the base and the base may reflect UV energy from the reflective surface in the base and reach different angles and sides of objects placed on the base support during the sanitization process.

According to the various embodiments, a sanitization unit with an active sanitizer base may include a UV light source in the base. The UV light source may be attached to the base on or near the reflective surface and under the support. UV light emitted from the UV light source may reflect from the reflective surface to reach different angles and sides of an object that is being sanitized and which is placed on the base support. According to an embodiment, a sanitization unit with an active sanitizer base may include UV light sources in both the base and the cover. According to the various embodiments, both the base and the cover may include a reflective surface.

According to the various embodiments, power from a power source may reach the sanitization unit through a power cord, and or onboard rechargeable batteries. According to an embodiment, the sanitization unit may include one power cord attached to either the base or the cover. According to a further embodiment, more than one power cord may be used to transmit power from a power source to either the cover and the base or both. In still another embodiment, both the base and cover may have rechargeable batteries that power UV sources in each.

According to the various embodiments, the base and cover of a sanitization container may be hindegly or removably attached to one another.

### BRIEF DESCRIPTION OF THE DRAWINGS

The accompanying drawings, which are incorporated herein and constitute part of this specification, illustrate exemplary embodiments of the invention, and, together with the general description given above and the detailed description given below, serve to explain the features of the invention.

FIG. 1 illustrates a perspective view of a horizontally disposed sanitization unit according to an embodiment.

FIG. 2 illustrates a cross sectional view of a horizontally disposed sanitization unit with a passive sanitizer base according an embodiment.

FIG. 3 illustrates a perspective view of a horizontally disposed sanitization unit with an active sanitizer base according an embodiment.

FIG. 4 illustrates a cross sectional view of a horizontally disposed sanitization unit with an active sanitizer base according to an embodiment.

FIG. 5 illustrates a cross sectional view of a horizontally disposed sanitization unit with an active sanitizer base with two power cords according to an embodiment.

FIGS. 6A and 6B illustrate cross sectional views of a horizontally disposed sanitization unit with active sanitizer bases with one power cord according to an embodiment.

FIGS. 7A and 7B illustrate different power configurations of various embodiments.

FIG. 8 illustrates a perspective view of a horizontally disposed sanitization unit with active sanitizer bases which is hingedly connected to the cover according to an embodiment.

FIG. 9 illustrates a cross sectional view of a horizontally disposed sanitization unit according to an embodiment.

FIG. 10 illustrates a perspective view of a horizontally disposed sanitization unit according to an embodiment.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes, and are not intended to limit the scope of the invention or the claims.

The word “exemplary” is used herein to mean “serving as an example, instance, or illustration.” Any implementation described herein as “exemplary” is not necessarily to be construed as preferred or advantageous over other implementations.

The method and apparatus of the various embodiments provide a convenient, compact, and easy to use sanitization unit which may be used to sanitize objects, such as keyboard or computing devices, from multiple directions and angles. According to the various embodiments, the sanitization unit may include reflective surfaces placed in the base or cover of a sanitization unit to allow UV radiation emitted from a UV light source to reflect off the reflectors and reach different sides and angles of an object that is placed inside the sanitization unit.

According to the various embodiments, the sanitization unit may include an ultraviolet (UV) light source particularly in the 200 to 300 nm wavelength range. In an embodiment, the UV light source has a 254 nm wavelength. This UV wavelength range has long been known for its germicidal and sanitization effects achieved by direct radiation. It is also well known that UV radiation in the 200 nm range can produce small quantities of ozone from oxygen in the atmosphere. Ozone, in sufficient concentrations is known to have significant germicidal and sanitization effects. Further, ozone, as a gas, is able to reach certain places and crevices such as beneath the keys and in crevices and out-of-sight surfaces that might exist on an object where direct UV radiation might not reach.

According to the various embodiments, the UV light source may be mounted within a sanitization unit such that the sanitization unit may expose UV radiation to different surfaces of objects placed inside of the unit. In an embodiment, the interior of the sanitization unit may be equipped with a UV light source and reflecting surface such that it directly and indirectly emits or reflects UV radiation to reach all parts of the object.

The UV light of the sanitization unit may be mounted in a number of configurations. Different UV light mounting configurations are disclosed in U.S. Pat. No. 6,458,331, the entire content of which is hereby incorporated by reference. For example, where an object, such as a keyboard or other input devices, is vertical or nearly so, as in the case of certain ATM machines and keypad access devices, the sanitization unit may be disposed vertically. The UV lamp may be a ring type lamp, a tubular UV lamp source that may be suspended horizontally in the sanitization container, a series of UV lamps that may be disposed around the perimeter of the sanitization container thereby directing radiation inward to the surfaces that are contained vertically or horizontally within the sanitization container.

The sanitization unit may be constructed to stand vertically for storage purposes. Examples of vertical designs of the sanitization unit are disclosed in the U.S. Design Pat. No. D616,563, the entire content of which is hereby incorporated by reference. The sanitization unit may also be constructed to stand horizontally. For example, the sanitization unit may be disposed horizontally and receive objects that may be placed on the support of the base. In this instance, a UV light source could be disposed horizontally within the sanitization unit with UV radiation shining downward or upward. In an exemplary embodiment, UV light sources may be mounted in a portion of the sanitization unit which is closed over or placed over the object (i.e., sanitization unit cover) allowing UV light to shine downward over any exposed surfaces where microbes might exist. Alternatively, the UV radiation could come from both above and below of an object (i.e., from both base and cover of a sanitization unit) to maximize the exposed surfaces thereby having fewer surfaces within shadows, consequently maximizing the surfaces that are exposed to the sterilizing, germicidal UV rays.

According to the various embodiments, the sanitization unit may include a sanitization chamber comprising a base which may be hingedly or removably attached to a cover. Placing the cover on the base may create a space/chamber inside of the sanitization unit in which objects may be contained for UV radiation. This will allow the sanitization unit to safely sanitize objects placed inside of the unit without exposing the outside environment to UV radiation.

In an embodiment, the sanitization unit further comprises a sanitization switch which is biased in the “off” position. When an object is placed inside of the sanitization unit and the unit is closed by placing the cover on top of the base (or bringing the base and cover together in a vertical sanitization unit), the sanitization switch may be engaged, either manually or automatically, and the UV radiation light source may be turned on. When the sanitization unit is opened (i.e., cover is removed), the UV radiation is immediately turned “off” as soon as the cover is removed or lifted.

In an embodiment, the sanitization unit may include a safety switch which, when engaged, may activate the UV light. Such a safety switch may be engaged automatically when the cover of the sanitization unit is closed. In this scenario, the safety switch may be designed in a way that the weight of the cover can turn “on” the UV light. Similarly, the safety switch may be disengaged automatically when the cover is lifted. Thus, once the weight of the cover is lifted from the switch, the UV light may turn “off.” Consequently, the UV light only turns on when the cover is on. This prevents exposure of the surrounding environment to UV radiation.

According to the various embodiments, a timer/power circuit for the UV light source may also be included. The timing/power circuit is activated as soon as the cover of the sanitization container is placed over the base and the sanitization switch is engaged. The timer may allow the UV light source to remain “on” for a predetermined amount of time. This time is consistent with substantial sanitization of the enclosed object. When the predetermined amount of time has expired, the UV light source may be turned “off” thereby saving both power as well as prolonging the life of the UV light source(s). Further, this limited time illumination minimizes any chemical breakdown of plastics that might occur due to UV radiation. In the event that the cover is lifted, the timer may be reset and, upon closing of the cover, the sanitization time period may begin again. According to the various embodiments, the length of time the timer may be activated to turn “on” and “off” the UV light source may be based on user preference. For example, a user may set the timer to allow for different UV illumination times keyed to the pathogens that a user may want to destroy.

According to the various embodiments, the sanitization unit may include an indicator light whereby, when sterilizing, the indicator light is lit. When sanitization is not in progress, such as when the sanitization lamp has burned out, the indicator light may not be lit. When the indicator light is “off,” the user may be informed that sanitization process is not in progress and may attend to the problem accordingly. Further, the indicator light may glow in different shades or colors noting when the unit is charged sufficiently for the next UV sanitizing cycle to begin.

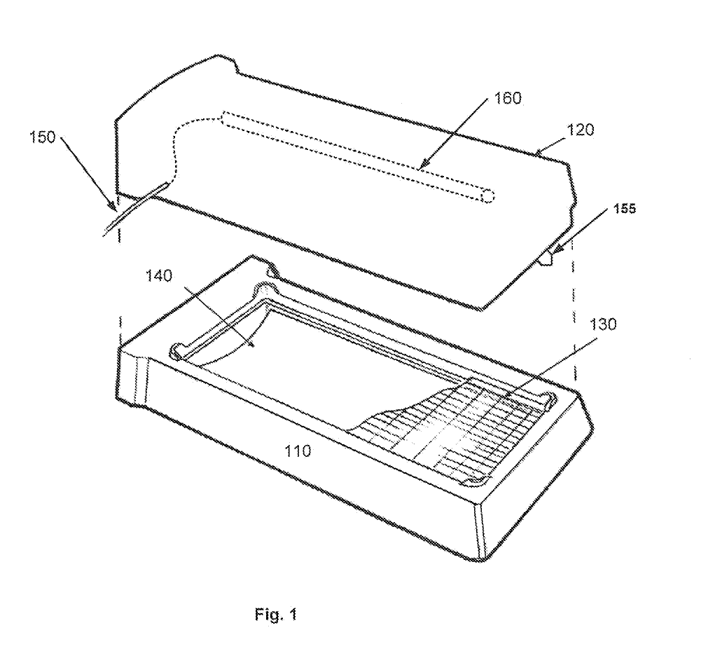

In an embodiment as shown in FIG. 1, a horizontally disposed sanitization unit with a passive sanitizer base may include a sanitization chamber formed when a passive sanitizing base 110 and an active sanitizing cover 120 are brought together. In this illustration, the active sanitizing cover may fit on top of the base 110. The base 110 may include a reflective surface 140 capable of reflecting UV light. The reflective surface 140 may be designed or shaped in different manners to allow it to reflect UV radiations in a diffuse or specular manner. In an exemplary embodiment, the shape of the reflective surface 140 may be convex to assist in reflection of UV radiation to different sides of an object that is placed in the sanitization container.

The reflective surface 140 may be constructed from any material that may allow or facilitate reflection of UV radiation. In an exemplary embodiment, the reflective surface 140 may be constructed using metals, such as aluminum, although this is not meant as a limitation. Aluminum reflective surfaces 140 may allow for reflection of UV radiation from the active UV cover to different sides of an object placed inside of the sanitization unit.

The base 110 may also include a support 130, in this case, illustrated in the form of a screen that may, removably, fixedly or hingedly, be placed in the base in a manner to cover the reflective surface 140. Objects that are to be sanitized may be placed inside of the sanitization chamber on top of the support 130. The support 130 may be a structure that may allow transmission of UV light, such as a screen (as shown in FIG. 1). Similarly, the support 130 may be made from a substance that may allow UV light transmission such as glass, quartz, or UV transmissive plastic.

The sanitization unit may be connected to an external power source via a power cord 150. In an embodiment, the sanitization container may include an internal power source (not shown), such as rechargeable batteries. When the sanitization unit includes an internal power source, such as batteries, the sanitization unit may not include a power cord 150, but may have a charging unit instead (not shown). Alternatively, the sanitization unit may include both an internal power source and power cord 150, such that when the internal power source is exhausted, the sanitization unit may use power from an external power source to operate and recharge the internal power source.

The sanitization unit 100 may receive power from a typical wall outlet via a power cord 150, which may be connected to an interlocking sanitization power switch 155. In an embodiment, power for the UV light source 160 (shown in phantom) may be received through the power cord 150 via the cover 120. The UV light source 160 may be mounted in the cover so as to direct UV illumination in a generally downward direction when the cover is oriented horizontally. The sanitization power switch 155 may be biased in the “off” position so that when the cover 120 is removed from the base 110 the UV light source 160 is turned “off.” Conversely, when the cover 120 is placed over the base 110, sanitization power switch 155 is “on” and power is provided to the lamps 160 and the lamps 160 may generate UV light (i.e., illumination). Further, when all UV light sources 160 are lit, an indicator light 165 may also light showing anyone viewing the apparatus that UV radiation is being generated by the UV light source 160 contained in the active UV sanitization container 120. It should be noted that the position of the indicator light 165 is entirely arbitrary and can be placed anywhere on the apparatus to provide satisfactory convenient viewing by the user.

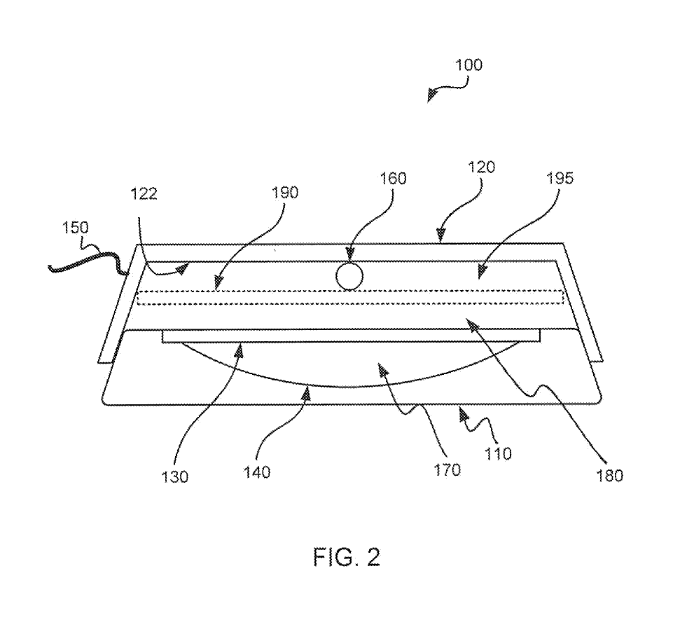

Referring now to FIG. 2 a cross section a passive sanitizing unit is illustrated. In an embodiment, the sanitization unit 100 with a passive sanitizer base may comprise a passive sanitizer base 110 including a reflective surface 140 which may receive and reflect UV radiation from a UV light source 160 (shown in FIG. 2) located in the active UV illumination cover 120. Once activated, light from the UV light generated by the UV light source 160 located in the active UV illumination cover 120 may be emitted down on objects placed on the support 130 of the passive UV base 110. The UV radiations from the UV light source 160 may reflect from the reflective surface 140 located on the base 110. When an object is placed inside the sanitization unit with a passive sanitation base, UV radiation may reach other sides of the object as it reflects off of the reflective surface 140. For example, with UV light sources 160 located in the active UV illumination cover 120, the sanitization unit 100 is able to sanitize the top, sides and bottom of an object placed inside of the sanitization unit 100. This configuration enhances the ability for all sides of an object to be illuminated by germicidal UV rays, but also may more effective use of the UV illumination since it is not being absorbed by a surface on which the active UV cover may sit when acting alone.

In an embodiment as illustrated in FIG. 2, a sanitization unit 100 with a passive sanitizer base 110 may include a reflective surface 140 and a support 130 placed over the reflective surface 140. The sanitization container 100 may also include a cover 120 with an interior surface 122. The sanitization unit 100 may be closed by placing the cover 120 on top of the base 110 thereby mating the cover 120 and base 110 to allow for sanitization of objects within a chamber, formed by the cooperation of the cover 120 and base 110, in the space between the active UV illumination cover and passive UV illumination base. A UV light source 160 may be removably or fixedly attached to the interior surface 122 of the cover 120. The number of UV light sources 160 shown is not meant as a limitation. The number of UV light sources 160 may vary depending upon the size of the lamp or the available space under the cover 120. Further, although the UV light source 160 is shown as individual tube type lamps, alternative shapes are well within the state-of-the-art including U-shaped lamps, ring-shaped lamps, individual bulb-type lamps, and indeed any other lamp that will emit the appropriate UV radiation necessary for the sanitization.

The base 110 may include a reflective surface 140 and a support 130. The support 130 may be placed on the base 110 over the reflective surface 140 in a manner to create a first space 170. When the cover 120 is placed on the base 110, a second space 180 may be created. Objects may be placed on the support 130 in the sanitization unit and contained in the second space 180 when the cover 120 is closed. It is understood that the size or dimensions of the second space 180 can vary.

In a further embodiment, the sanitization unit 100 may include a protective cover 190 to protect the UV light source 160. For example, the protective cover 190 may prevent contact between the UV light source 160 and an object that may be placed in the sanitization unit. The protective barrier 190 may be in a form of a partition, screen or other UV transmissive material in the manner such as shown in FIG. 2. When the protective barrier 190 is in the form of a partition, it may create a third space 195 in which the UV light source 160 may be located. The protective cover 190 may also include any type of covering that surrounds the UV light source 150 to protect it from being damaged while sterilizing objects. The protective cover 190 may be a screen which may allow UV light to pass through to reach an object placed in the sanitization unit. The protective barrier 190 may also be made from a material that may allow transmission of UV light, such as glass or plastic. The inside surface of the active UV illumination cover may comprise UV reflective surface(s) such as aluminum or other UV reflective surfaces. Further, the active UV illumination cover may further comprise an additions curve convex reflective surface similar to that illustrated as 140, except resident in the cover. Again, reflective surfaces may be diffuse or specular in nature.

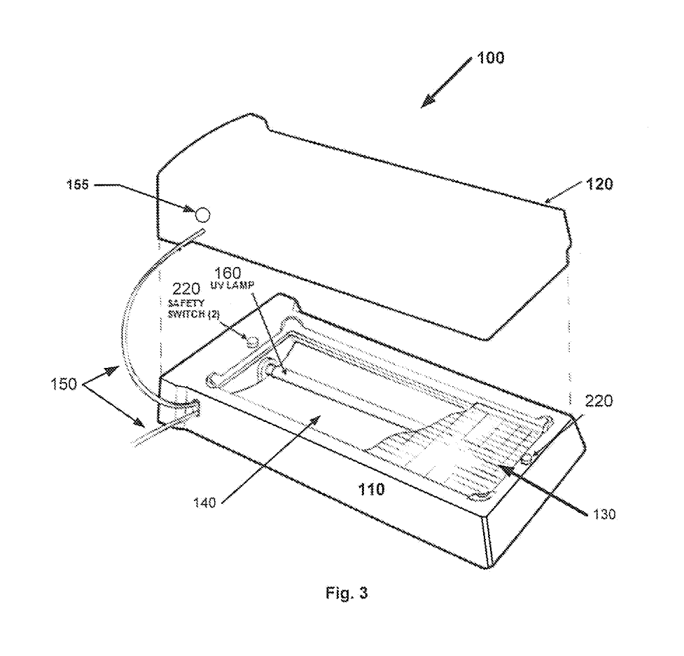

FIG. 3 illustrates a sanitizing container 100 with an active sanitizer base 110 which includes a UV light source 160 placed in the first space between the support 130 and the reflective surface 140. The UV light source 160 may sanitize the side of an object placed on the support 130 that faces the UV light source 160. Reflection of the UV radiations off of the reflective surface 140 may also allow for sanitization of different sides and angles of the object that is placed on the support 130.

In an exemplary embodiment, when the sanitization container 100 includes an active sanitizer base, the cover 120 may or may not include a UV light source 160. In such a scenario, the cover 120 may also include a reflective surface (not shown) to allow reflection of UV radiations to sanitize different sides of an object placed on the support 130 of the sanitization container 100.

The sanitization container 100 with an active sanitizer base may be controlled by a sanitization power switch 155. When in the “on” position, the UV light source 160 may be activated to sanitize objects placed in the sanitization unit. In an embodiment, the sanitization power switch 155 may only be functional when safety switches 220 are engaged. The safety switches 220 may be located on the base 110 in such a manner that when the cover 120 is placed on the base 110 to close the sanitization container 100 the safety switches 220 may be engaged. This provides a safety mechanism by which UV light sources 160 may only be activated when the cover 120 is placed on the base 110. When the cover 120 is off (i.e., when the UV light sources are exposed to the environment) and the safety switches 220 are disengaged, UV light sources may not be activated. The safety switches 220 may operate mechanically or electronically. For example, the safety switches 220 may include push down switches which may be engaged (i.e., pushed down) when the cover 120 is placed on top of them. The safety switches 220 may also include a laser sensor mechanism which may require a receptor sensor in the cover 120 to be placed near the laser sensors in the base 110 before they are engaged. Mechanism and types of mechanical and electronic switches are well known and may be included in the various embodiments. In an alternative embodiment, the safety switches 220 may be located on the cover 120.

In an embodiment, the active cover and active base may be separate controlled by their own power switches. Further, the placement of the safety switches is for illustrative purposes only. The number of switches and their location can be varied to afford the appropriate safety.

In an embodiment, the sanitization container 100 with an active sanitizer base may include power cords 150 which may supply power to both the UV light sources in the base 110 and the cover 120. Accordingly, more than one power cord 155 may be present. Alternatively, only one power cord 150 may supply power to either the base 110 or cover 120 UV light sources 160 and power is transferred from one unit to the other through mechanisms other than a power cord 150. For example, when each of the base 110 and cover 120 require power to activate UV light sources 160, one power cord 150 may be directed to the base 110. In such a scenario, power received in the base 110 may be conducted to the UV light source 160 located in the cover 120 through electrodes in the cover 120 that may come into contact with electrodes in the base 110 when the sanitization container 100 is closed by placing the cover 120 on top of the base 110.

It should also be noted that a surprising additional benefit from having both an active cover and and an active base is that the amount of the UV illumination is delivered in a much more effective fashion and more quickly. Thus, effective amounts of UV illumination required to kill harmful bacteria and viruses is delivered more effectively. For example, energy from the UV lamp 160 and the corresponding UV lamp in the active cover will provide illumination to the reflective surfaces of the opposite structure. For example, UV illumination from the active cover 120 will provide illumination to objects being sanitize that are placed on support 130. However, in addition, the illumination from the active cover 120 will be reflected from the reflecting surface 140 in the active base unit. Thus, energy will not be wasted, but rather will be delivered to all locations on the surface of objects being sanitized. Similarly, UV from the UV lamp 160 in the active base will be reflected off the reflecting surface in the active cover 122 again impinge on objects resting on support 130 from all different angles. Thus, the unit does not waste any UV energy and is therefore more effective.

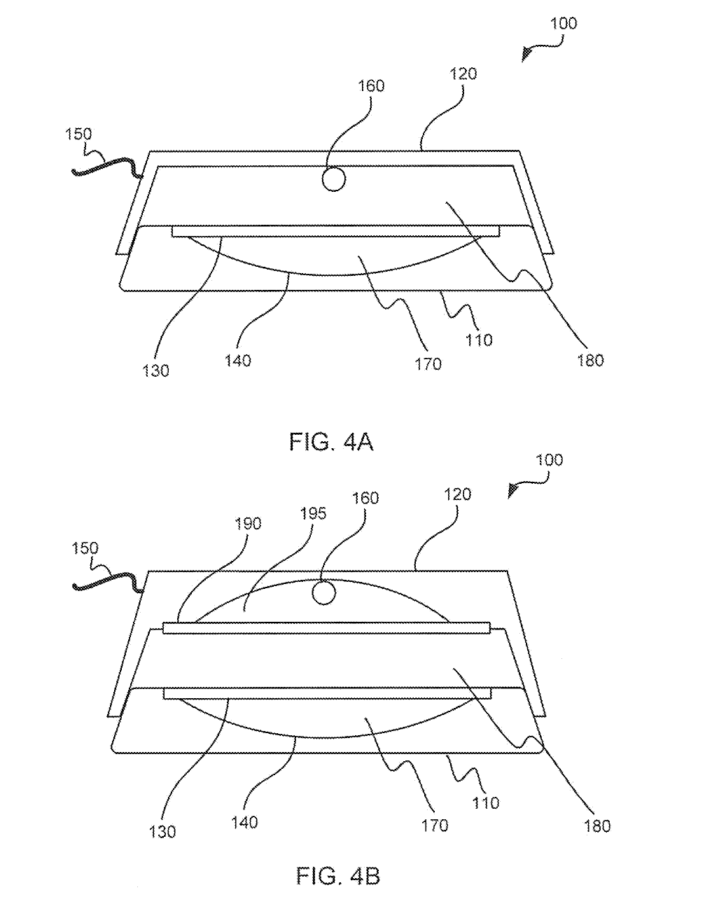

FIGS. 4A and 4B illustrate an exemplary embodiment sanitization container 100 with an active sanitization cover 120 and a passive base 110. The sanitization container 100 may include a cover 120 comprising a UV light source 160. The passive base one can may comprise a reflective surface 140 and a support 130. When the container 100 is closed, upon turning “on” the power, UV light radiation may be emitted from the UV source 160 to illuminate the surface of an object to be sanitized that is facing the UV source 160. The cover 121 placed over the base 110 creates a space 180 that provide sufficient space for objects to be placed on support 130 to be illuminated with UV radiation. UV illumination that reaches the reflecting surface 140 will tend to be reflected back in an upward direction to sanitize the surfaces of an object to be sanitized that is resting on the support 130 but which surfaces are facing the reflective surface 140. Reflections of the UV radiation from the reflective surfaces 140, may reach different sides and angles of the object during the sanitization process. The space created between the cover 130 and the reflecting surface 140 provides space for the reflection of UV energy from source 162 be reflected on to those surfaces of an object facing reflecting surface 140.

Referring now to FIG. 4B, yet another embodiment of an active cover and a passive base is illustrated. In this case, the active cover further comprises a reflecting surface 142 that reflects UV radiation from UV source 160 in a downward direction. A protective barrier 190 provides protection for the UV source while at the same time allows UV radiation to be transmitted through the protective barrier 190 into the space 180 it is created between the active cover 120 and the passive base 110. The space 180 provides room for objects to be placed on support 130 in order to be sanitized.

Protective barrier 190 may comprise, without limitation, the screen, a UV transparent material such as a plastic, quartz, or other material known in the art to transmit significant amounts of UV radiation.

FIG. 4B has the same characteristics as noted with respect to FIG. 4A above.

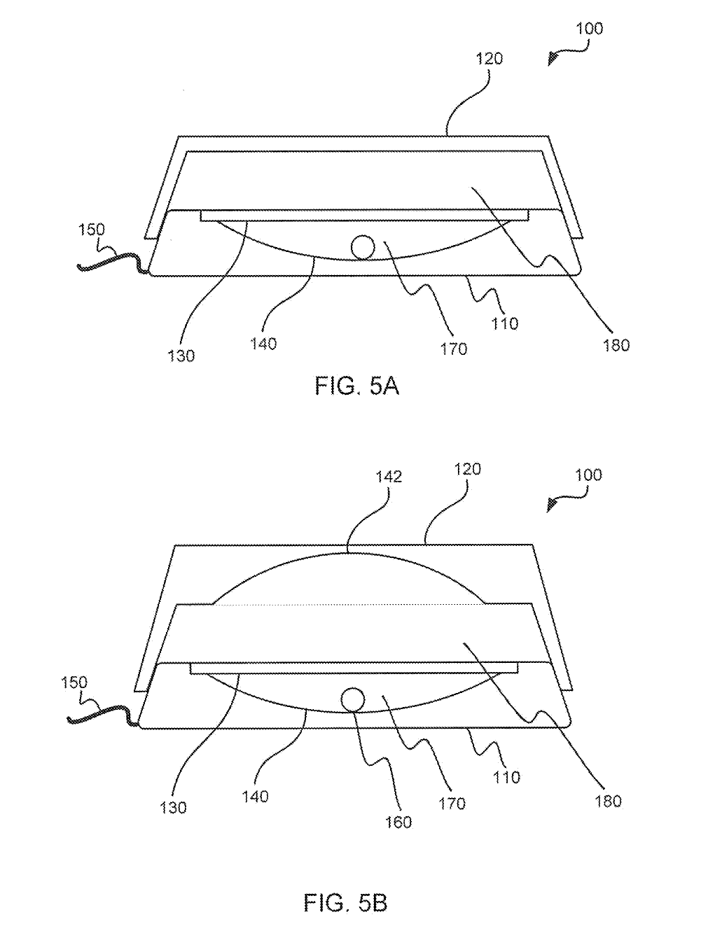

FIGS. 5A and 5B illustrate an exemplary embodiment sanitization container 100 with an active sanitization base including UV light sources 160 in the base 110 and a cover 120 that does not have a UV illumination source. The container 100 may also include reflective surfaces 140 in the base and reflective surface 142 in the cover. The additional reflective surface 142 in the cover may allow UV radiation emanating from the UV source 60 in the active base to be reflected from above an object to be sanitized that is resting on support 130 and therefore to reach different angles and sides of an object during the sanitization process. In this fashion, UV energy is conserved and more effectively used for the sanitizing process.

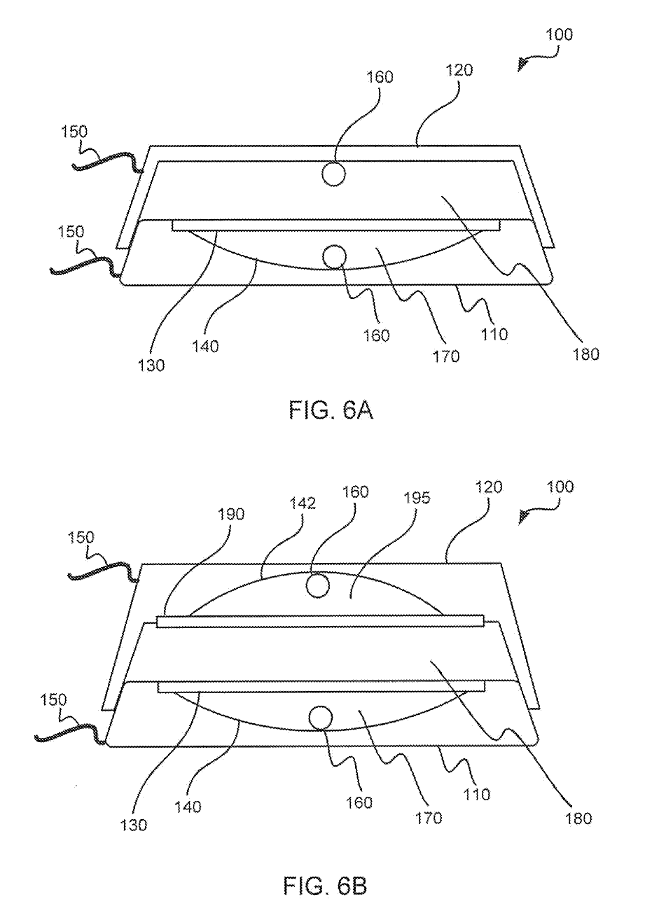

As illustrated in FIGS. 6A and 6B, power may reach the sanitization units 100 through power cords 150 which are connected to both the base 110 and the cover 120. In a further embodiment, as illustrated in FIG. 6A, the power may be transmitted only to the base 110 through one power cord 150. The base 110 may then transmit power to the cover 120 through a second power cord. In a further embodiment, the power may be transmitted only to the cover 110 through one power cord 150. The cover 120 may then transmit power to the base 110 through a second power cord. In FIG. 6A, the cover 120 may have a reflecting surface 121 illustrated in FIG. 6B, 142. In this embodiment, the internal surface 122 of cover 120 may be coated with a reflecting material to reflect UV throughout space 180 onto objects placed on support 130. Space 180 provides room to place objects on support 130 for sanitizing. UV lamp 160 is present in both the cover 120 and the base 110 to provide active UV sanitizing. In the base 110 there is space 170 between the UV source 160 and the support 130 to allow for appropriate reflection of UV energy from the source 160. Thus, both direct and reflected UV energy impinges on any object to be sanitized the displaced on support 130. In FIG. 6B, a cover unit having a reflector 142 is illustrated. In this instance, a protecting shield 190 is illustrated to protect lamp 160 from damage but still allow for transmission of UV light from lamp 160. Space 180 allows objects to be placed therein for sanitizing. Other structures are similar to those illustrated in FIG. 6A.

It should be noted that the embodiments noted in FIGS. 6A and 6B have the surprising result of more effectively sanitizing objects that are placed in spaces 180 since UV energy from the base UV source 160 reflects not only from reflector 140, but also from reflector surface 122. Similarly UV from the cover source 160 is reflected from reflector 140 as well as its own reflector 142 thereby not wasting any UV energy through absorption. Thus, a much more efficient use of UV energy is created in all embodiments illustrated herein

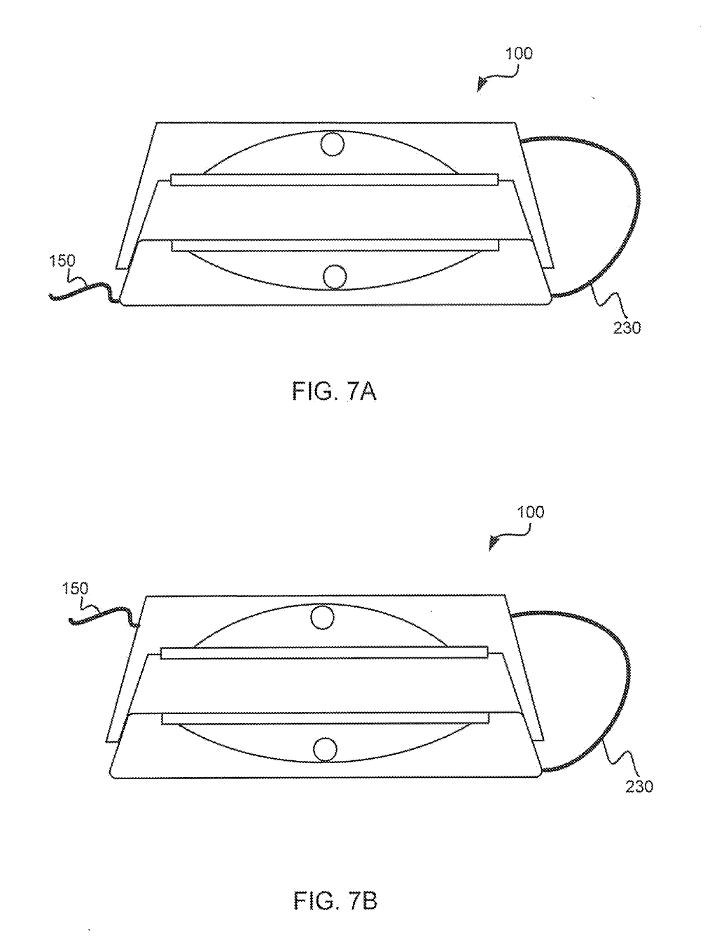

FIGS. 7A and 7B illustrate alternate embodiments for powering embodiments having active sanitizing bases and covers. In FIG. 7A, the “master” power comes to the combined unit via power cord 150 through the lower reflecting base 110 which is an active base having its own UV illumination source. Power is then distributed to the cover 120 via power cord 230. Cover 120 is an active cover with its own UV source. In FIG. 7B, the master power comes in through the cover 120 which, in this illustration, is an active cover having its own UV source and is distributed to the base 110 via power cord 230. In this instance, base 110 is an active base having its own UV source.

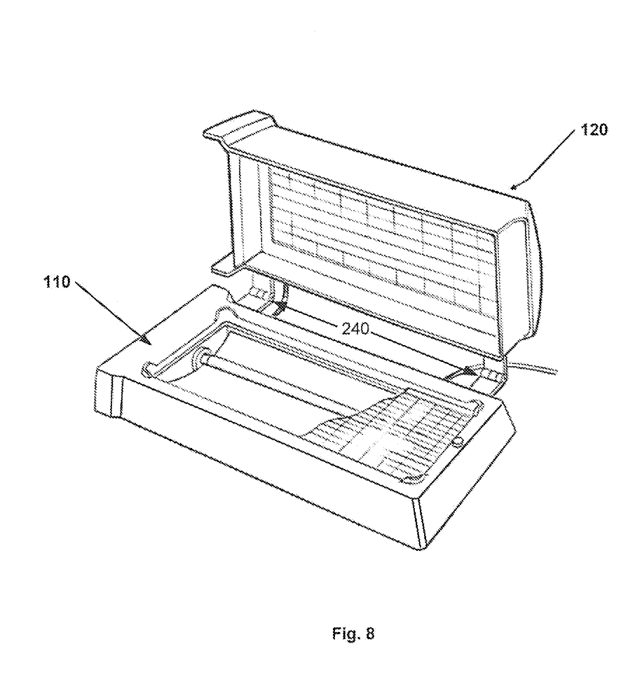

FIG. 8 illustrates an exemplary embodiment sanitization unit with an active sanitizer base 110 which is hingedly attached to an active cover 120 using hinges 240. Hinges 240 are well known and different types of hinges 240 or hinge type mechanisms are contemplated.

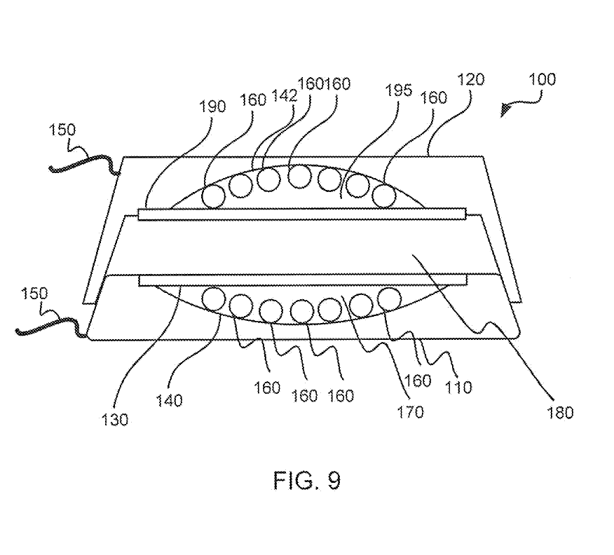

FIG. 9 illustrates an exemplary embodiment sanitization unit similar to the sanitization unit described above with reference to FIG. 6B, except that rather than a single UV lamp (i.e., light source) 160, a plurality of UV lamps 160 may be present in both the cover 120 and the base 110. In an embodiment, the arrangement of the plurality of UV lamps 160 may follow the curve of the reflecting surfaces 142.

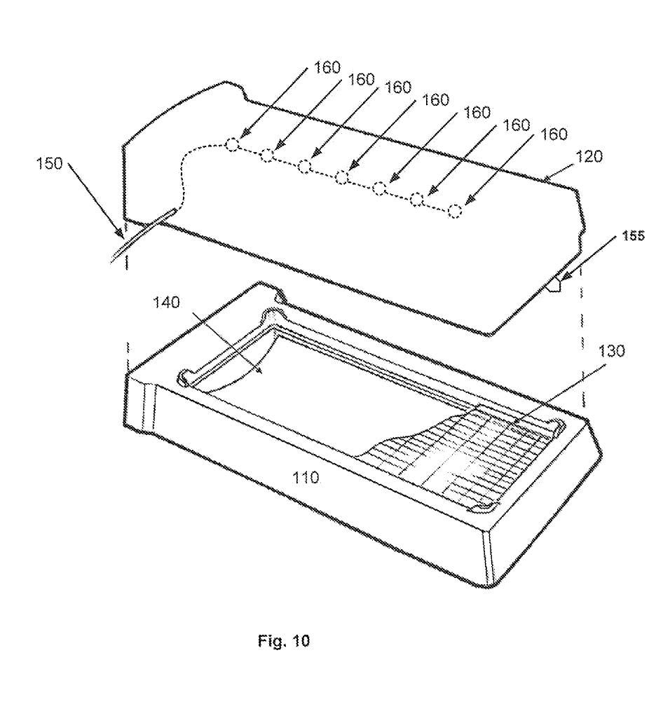

FIG. 10 illustrates an exemplary embodiment sanitization unit similar to the sanitization unit described above with reference to FIG. 1, except that rather than a single UV light source (i.e., lamp) 160, a plurality of UV light sources (i.e., lamps) 160 (shown in phantom) may be attached to the cover 120. In an embodiment, the plurality of UV light sources 160 may be an arrangement (e.g., string, cluster, parallel line, etc.) of UV light sources 160, such as an arrangement of light emitting diodes (LEDs), mercury vapor bulbs, fluorescent bulbs, etc.

The sanitization units of the various embodiments may include one or more UV light sources disposed in the cover and/or base in various arrangements, and the number and/or arrangement of UV light sources may be different in the cover and the base. In the various embodiments the one or more UV light sources may be any type light source configured to output UV radiation during operation, such as LEDs, mercury vapor bulbs, florescent bulbs, etc.

The sanitization unit of the various embodiments may be used for sanitization of different objects. For example, the sanitization unit of the various embodiments may be used to sanitize medical instruments, electronic devices (e.g., keyboards or input devices), laboratory instruments, or any other tools, instruments, infant objects, eating utensils, or any other objects that that may require sanitization. Dimensions of the unit may be variable and are driven by the size of the objects intended to be sanitized. Thus, no specific dimensions are provided herein, but will be appreciated as being flexible by those of ordinary skill in the art.

UV illumination time is driven by the amount of UV illumination required for destruction of certain pathogens. Quantities of UV illumination in micro-watt-seconds per square centimeter are well known. Thus, wattage and UV sources and time of illumination may be varied to deliver the necessary dosage.

It will also be understood that the invention may be embodied in other specific forms without departing from the scope of the invention disclosed and that the examples and embodiments described herein are in all respects illustrative and not restrictive. Those skilled in the art of the present invention will recognize that other embodiments using the concepts described herein are also possible. Further, any reference to claim elements in the singular, for example, using the articles “a,” “an,” or “the” is not to be construed as limiting the element to the singular.

What is claimed is:

1. A sanitization unit, comprising:
a cover, the cover comprising a first ultraviolet (UV) light source mounted in the cover so as to direct UV illumination in a generally downward direction when the cover is oriented horizontally; and
a base, the base comprising:
a second UV light source mounted in the base so as to direct UV illumination in a generally upward direction when the base is oriented horizontally; and
a support, the support comprising UV transmissive material and configured to support an object placed in the sanitization unit,
wherein the cover is configured to mate with the base such that the cover and base cooperate to form a chamber configured to encompass the object placed in the sanitization unit and to prevent UV illumination from radiating out of the chamber.

2. The sanitization unit of claim 1, wherein the first UV light source and the second UV light source are one or more of tube type lamps, U-shaped lamps, ring-shaped lamps, and individual bulb-type lamps.

3. The sanitization unit of claim 1, wherein the first UV light source and the second UV light source are one or more of light emitting diodes, mercury vapor lamps, and fluorescent lamps.

4. The sanitization unit of claim 1, wherein the first UV light source and the second UV light source are configured to generate UV illumination with a wave length of 200 to 300 nanometers.

5. The sanitization unit of claim 1, wherein the cover includes a first reflective surface.

6. The sanitization unit of claim 5, wherein the base includes a second reflective surface.

7. The sanitization unit of claim 6, wherein the first reflective surface and the second reflective surface are made from aluminum.

8. The sanitization unit of claim 7, wherein the first reflective surface and the second reflective surface are convex shapes.

9. The sanitization unit of claim 1, wherein the support is a screen.

10. The sanitization unit of claim 9, wherein the support is made from a material selected from a group consisting of glass, quartz, and plastic.

11. The sanitization unit of claim 1, wherein the first UV light source and the second UV light source each are a plurality of lamps.

12. A sanitization unit, comprising:
a cover; and
a base, the base comprising:
a first UV light source mounted in the base so as to direct UV illumination in a generally upward direction when the base is oriented horizontally; and
a support, the support comprising UV transmissive material and configured to support an object placed in the sanitization unit,
wherein the cover is configured to mate with the base such that the cover and base cooperate to form a chamber configured to encompass the object placed in the sanitization unit and to prevent UV illumination from radiating out of the chamber.

13. The sanitization unit of claim 12, wherein the first UV light source is one or more of a tube type lamp, U-shaped lamp, ring-shaped lamp, and individual bulb-type lamp.

14. The sanitization unit of claim 12, wherein the first UV light source is one or more of light emitting diodes, mercury vapor lamps, and fluorescent lamps.

15. The sanitization unit of claim 12, wherein the first UV light source is configured to generate UV illumination with a wave length of 200 to 300 nanometers.

16. The sanitization unit of claim 12, wherein the base includes a first reflective surface.

17. The sanitization unit of claim 16, wherein the cover includes a second reflective surface.

18. The sanitization unit of claim 17, wherein the first reflective surface and the second reflective surface are made from aluminum.

19. The sanitization unit of claim 18, wherein the first reflective surface and the second reflective surface are convex shapes.

20. The sanitization unit of claim 12, wherein the support is a screen.

21. The sanitization unit of claim 20, wherein the support is made from a material selected from a group consisting of glass, quartz, and plastic.

22. The sanitization unit of claim 1, wherein the first UV light source is plurality of lamps.

23. A sanitization unit, comprising:
a cover, the cover comprising a first ultraviolet (UV) light source mounted in the cover so as to direct UV illumination in a generally downward direction when the cover is oriented horizontally; and
a base, the base comprising a support, the support comprising UV transmissive material and configured to support an object placed in the sanitization unit,
wherein the cover is configured to mate with the base such that the cover and base cooperate to form a chamber configured to encompass the object placed in the sanitization unit and to prevent UV illumination from radiating out of the chamber.

24. The sanitization unit of claim 23, wherein the first UV light source is one or more of a tube type lamp, U-shaped lamp, ring-shaped lamp, and individual bulb-type lamp.

25. The sanitization unit of claim 23, wherein the first UV light source is one or more of a light emitting diode, mercury vapor lamp, and fluorescent lamp.

26. The sanitization unit of claim 23, wherein the first UV light source is configured to generate UV illumination with a wave length of 200 to 300 nanometers.

27. The sanitization unit of claim 23, wherein the cover includes a first reflective surface.

28. The sanitization unit of claim 27, wherein the base includes a second reflective surface.

29. The sanitization unit of claim 28, wherein the first reflective surface and the second reflective surface are made from aluminum.

30. The sanitization unit of claim 29, wherein the first reflective surface and the second reflective surface are convex shapes.

31. The sanitization unit of claim 23, wherein the support is a screen.

32. The sanitization unit of claim 31, wherein the support is made from a material selected from a group consisting of glass, quartz, and plastic.

33. The sanitization unit of claim 23, wherein the first UV light source light source is a plurality of lamps.

34. A method for sanitizing an object, comprising:
enclosing the object in a chamber of a sanitization unit, wherein the chamber is formed by the mating of a base and a cover and the chamber is configured to encompass the object placed in the sanitization unit and to prevent ultraviolet (UV) illumination from radiating out of the chamber; and
sanitizing the object by generating UV illumination within the chamber of the sanitization unit.

35. The method of claim 34, wherein the UV illumination is generated by a UV light source mounted in the base so as to direct UV illumination in a generally upward direction when the base is oriented horizontally.

36. The method of claim 34, wherein the UV illumination is generated by a UV light source mounted in the cover so as to direct UV illumination in a generally downward direction when the cover is oriented horizontally.

37. The method of claim 34, wherein the UV illumination is generated by a first UV light source mounted in the base so as to direct UV illumination in a generally upward direction when the base is oriented horizontally and is generated by a second UV light source mounted in the cover so as to direct UV illumination in a generally downward direction when the cover is oriented horizontally.

38. The sanitization unit of claim 37, wherein the first UV light source and the second UV light source are one or more of tube type lamps, U-shaped lamps, ring-shaped lamps, and individual bulb-type lamps.

39. The sanitization unit of claim 37, wherein the first UV light source and the second UV light source are one or more of light emitting diodes, mercury vapor lamps, and fluorescent lamps.

40. The sanitization unit of claim 37, wherein the first UV light source and the second UV light source each are a plurality of lamps.
